(19) United States
(12) Patent Application Publication
(10) Pub. No.: US 2025/0034321 A1
(43) Pub. Date: Jan. 30, 2025

(54) COMPOSITIONS OF EPOXY CURING AGENT INCORPORATING NAPHTHOL AND NAPHTHOL DERIVATIVES

(71) Applicant: EVONIK OPERATIONS GMBH, Essen (DE)

(72) Inventors: Sudhir ANANTHACHAR, Hillsborough, NJ (US); Gauri Sankar LAL, Whitehall, PA (US); Nicholas BAURKOT, Easton, PA (US); Raghuraman GOVINDAN KARUNAKARAN, Macungie, PA (US); Shafiq FAZEL, Allentown, PA (US); Michael COOK, Nuneaton (GB)

(73) Assignee: EVONIK OPERATIONS GMBH, Essen (DE)

(21) Appl. No.: 18/696,880

(22) PCT Filed: Sep. 23, 2022
(86) PCT No.: PCT/EP2022/076482

§ 371 Date: Mar. 28, 2024

Related U.S. Application Data
(60) Provisional application No. 63/252,599, filed on Oct. 5, 2021.

(51) Int. Cl. C08G 59/62; C07C 49/747; C07C 215/50; C07D 241/04; C09D 163/00

(52) U.S. Cl. CPC C08G 59/623; C07C 49/747; C07C 215/50; C07D 241/04; C09D 163/00

(57) ABSTRACT

The present invention relates to epoxy curing agent compositions comprising naphthol and naphthol derivatives in combination with at least one polyamine having three or more active amine hydrogens, and use of these curing agents as hardener for epoxy resins. These curing agent compositions may be used to cure, harden and/or crosslink an epoxy resin.

### BACKGROUND OF THE INVENTION

Epoxy curing agents are used in a wide variety of applications in industry. These include industrial coatings and composite materials. Cured epoxy resin systems confer excellent adhesion, chemical resistance, good mechanical and electrical insulating properties and in some cases heat resistance to the finished product. They are particularly useful for protecting metal and concrete surfaces as well as cementitious and ceramic substrates.

In order to convert epoxy resins to hard, infusible thermoset networks it is necessary to use crosslinking agents. These cross linkers, hardeners or curing agents are widely known, promote cross-linking or curing of epoxy resins. Epoxy resins contain epoxy groups which react with amines, carboxylic acids and mercaptans to effect the cure. Curing can occur by either homo-polymerization initiated by a catalytic curing agent or a polyaddition/copolymerization reaction with a multifunctional curing agent.

Many industrial applications for epoxy coatings require a fast return to service for improved productivity. There is a market need for improved reactivity and performance at low temperatures e.g. 5° C. or in some cases as low as 0° C. The current epoxy curing systems that are used at these temperatures do not provide adequate coating performance. The cure is too slow and as a result the coatings suffer from defects such as blushing, carbamation and water spotting. This results from unreacted amine curing agent migration to the surface of the coating and reacting with atmospheric moisture and carbon dioxide forming a greasy white film (carbamation). In addition, the slow cure results in a longer time for the coating to dry or set and hence longer time to apply subsequent coats.

In order to increase the rate of cure of epoxy coatings at low temperatures, applicators have traditionally used epoxy accelerators. These include, tertiary amines, phenols and phenol derivatives such as Mannich bases, acids such as salicylic acid, p-toluenesulfonic acid and sulfuric acid. These accelerators can only be used at low levels and suffer from several disadvantages including brittleness of coatings due to the initiation of homo-polymerization of the epoxy resin. There are also increasing health and safety concerns associated with phenol and substituted phenols such as the toxicity and mutagenicity of this class of compounds. Indeed, there are increasing regulatory pressure by professional bodies and consumers on the use of phenol and phenol derivatives in coating materials.

There is a need in the art for curing agent compositions for epoxy resins which can accelerate the cure speed at sub-ambient temperature (e.g. 5° C.) and provide a safer alternative to currently used materials. We here-in disclose naphthol and naphthol derivative compositions which provide excellent cure speed while being safer to health and the environment.

### BRIEF SUMMARY OF THE INVENTION

This invention relates to epoxy curing agent compositions comprising naphthol and naphthol derivatives in combination with at least one polyamine having three or more active amine hydrogens, and use of these curing agents as hardener for epoxy resins. These curing agent compositions may be used to cure, harden and/or crosslink an epoxy resin. In addition, these inventive compositions can provide dry cure of epoxy coatings at ambient temperature (23° C.) or at 5° C. at much higher rate than the state of the art fast curing epoxy systems containing phenols or phenol derived Mannich bases and phenalkamines.

The faster curing epoxy curing systems of this invention provide the advantages of lower tendency to carbamate and shorter time for coatings to dry as compared to traditional epoxy accelerators such as phenol Mannich bases and salicylic acid. In addition, naphthol or naphthol derivatives in coating compositions do not degrade the flexibility of the coating and act as plasticizers in enhancing the extent of cure.

One aspect of the invention relates to a curing agent composition comprising
(a) at least one naphthol or naphthol derivative represented by the structure (1) below:

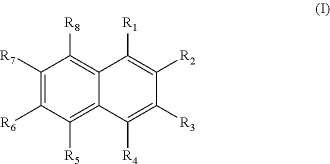

wherein R₁ and R₂ independently of each other=OH, H, C₁-C₁₀ alkyl, C₁-C₁₀ aryl, C₁-C₁₀ alkyl or aryl ether, NH₂, Cl, Br, I, NO₂, HSO₃, or X(CH₂)NHY with Y═C₁-C₁₀ alkyl, C₁-C₁₀ aryl, or a polyamine and X=Ph or C₁-C₄ alkyl; R₃-R₈═H, C₁-C₁₀ alkyl, C₁-C₁₀ aryl, C₁-C₁₀ alkyl or aryl ether, NH₂, Cl, Br, I, NO₂, or HSO₃; and wherein one of R₁ or R₂═OH; and
(b) at least one polyamine having three or more active amine hydrogens.

Preferably, in one embodiment, the at least one naphthol or naphthol derivative is represented by the structures (II)-(V) below:

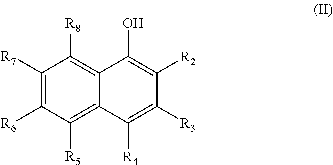

wherein R₂-R₈═H, C₁-C₁₀ alkyl, C₁-C₁₀ aryl, C₁-C₁₀ alkyl or aryl ether, NH₂, Cl, Br, I, NO₂, or HSO₃;

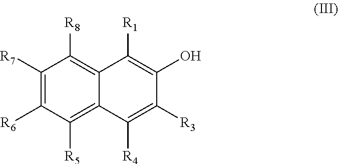

wherein R₁ and R₃-R₈═H, C₁-C₁₀ alkyl, C₁-C₁₀ aryl, C₁-C₁₀ alkyl or aryl ether, NH₂, Cl, Br, I, NO₂, or HSO₃;

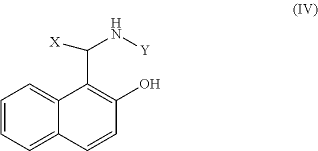

wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl; and

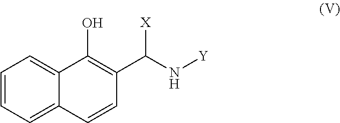

wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl.

The compounds of structures (II) and (Ill) can be synthesized by conventional methods or purchased from commercial sources while the naphthol derivatives of structures (IV) and (V) are obtained by a Mannich reaction whereby the 1-naphthol (α-naphthol) or 2-naphthol (β-naphthol) is reacted with an aldehyde and an amine to form a Mannich base.

Preferably, the polyamine compound used in the reaction with 1-naphthol or 2-naphthol may be an alkylene polyamine e.g. ethylenediamine, a polyalkylene polyamine e.g. diethylenetriamine, triethylenetetramine, tetraethylenepentamine, N¹-(3-dimethylaminopropyl)propylenediamine (DMAPAPA), dimethylaminopropylamine (DMAPA), an arylalkyl polyamine such as m-xylenediamine, a cycloaliphatic polyamine such as 4,4′-methylene-dicyclohexylamine (PACM), or a polyetherpolyamine such as Jeffamine D230.

Another aspect of the invention relates to a composition comprising naphthol and naphthol derivatives in combination with at least one polyamine having three or more active amine hydrogens and a multi-functional epoxy resin.

In the preparation of the compositions of this invention the naphthol or naphthol derivative can be dissolved into the polyamine prior to being contacted with the epoxy resin component. Alternatively, the naphthol or derivative can be dissolved into the resin and the mixture is then treated with the polyamine.

Preferably, in one embodiment curing agent compositions of the present disclosure have an amine hydrogen equivalent weight (AHEW) based on 100% solids from 50 to 500. The present disclosure, in another aspect, provides amine-epoxy compositions and the cured products produced therefrom. For example, an amine-epoxy composition, in accordance with the present disclosure, comprises the reaction product of a curing agent composition containing the novel compositions comprising at least one naphthol or naphthol derivative and having at least two active amine hydrogen atoms and epoxy composition comprising at least one multifunctional epoxy resin.

Preferably, in one embodiment the naphthol or naphthol derivative is 0.5-50 wt. % relative to the amine in the curing agent composition.

The present disclosure also provides for the use of a curing agent composition comprising a naphthol or naphthol derivative represented by the structures (I)-(V) and at least one polyamine having three or more active amine hydrogens as a hardener for epoxy resins.

Articles of manufacture are produced from amine-epoxy compositions disclosed herein including, but are not limited to, coatings, primers, sealants, curing compounds, construction products, flooring products, and composite products. Further, such coatings, primers, sealants, or curing compounds may be applied to metal or cementitious substrates. The mix of curing agent and epoxy resin component often requires no “ripening time” for obtaining contact products with high gloss and clarity.

Ripening time or incubation time is defined as the time between mixing epoxy resin component with amine and applying the product onto the target substrate. It could also be defined as the time required for the mix to become clear. Furthermore, the novel curing agent compositions also provide faster amine-epoxy reaction rate. These unique properties provide the advantages of lower tendency to carbamate and shorter time for coatings to dry as compared to traditional epoxy accelerators products derived from alkylene polyamines such as ethylenediamine and diethylenetriamine with phenols.

### DETAILED DESCRIPTION OF INVENTION

In the preparation of the compositions of this invention the naphthol or naphthol derivative can be dissolved into the polyamine prior to being contacted with the epoxy resin component. Alternatively, the naphthol or naphthol derivative can be dissolved into the resin and the mixture is then treated with the polyamine.

One aspect of the invention relates to a curing agent composition comprising (a) at least one naphthol or naphthol derivative represented by the structure (1) below:

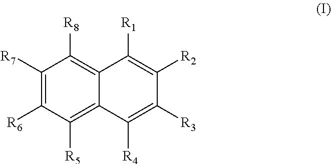

wherein R₁ and R₂ independently of each other=OH, H, C₁-C₁₀ alkyl, C₁-C₁₀ aryl, C₁-C₁₀ alkyl or aryl ether, NH₂, Cl, Br, I, NO₂, HSO₃, or X(CH₂)NHY with Y═C₁-C₁₀ alkyl, C₁-C₁₀ aryl, or a polyamine and X=Ph or C₁-C₄ alkyl; R₃-R₈═H, C₁-C₁₀ alkyl, C₁-C₁₀ aryl, C₁-C₁₀ alkyl or aryl ether, NH₂, Cl, Br, I, NO₂, or HSO₃; and wherein one of R₁ or R₂═OH; and
(b) at least one polyamine having three or more active amine hydrogens.

Preferably, in one embodiment, the at least one naphthol or naphthol derivative is represented by the structures (II)-(V) below:

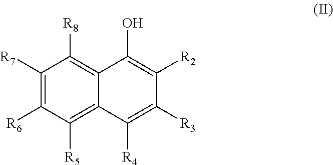

wherein R₂-R₈═H, C₁-C₁₀ alkyl, C₁-C₁₀ aryl, C₁-C₁₀ alkyl or aryl ether, NH₂, Cl, Br, I, NO₂, or HSO₃;

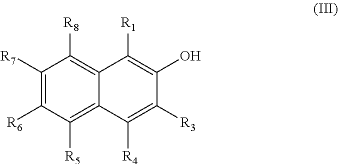

wherein R₁ and R₃-R₈═H, C₁-C₁₀ alkyl, C₁-C₁₀ aryl, C₁-C₁₀ alkyl or aryl ether, NH₂, C₁, Br, I, NO₂, or HSO₃;

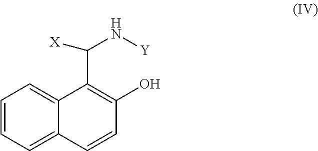

wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl; and

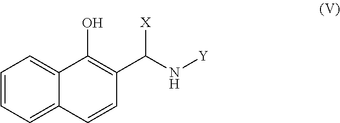

wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl.

The naphthol compounds of structures (II) and (III) can be synthesized by methods known in the art or purchased from commercial sources. Preferred examples of these compounds include 4-methyl-1-naphthol, 2-methyl-1-naphthol,4-amino-3-methyl-1-naphthol, 4-methoxy-1-naphthol, 3-methoxy-2-naphthol, 5-methoxy-1-naphthol, 4-chloro-1-naphthol,1-chloro-2-naphthol, 1-bromo-2-naphthol, 1-naphthol-4-sulfonic acid etc.

Preferably, in one embodiment the at least one naphthol compound is selected from the group consisting of 4-methyl-1-naphthol, 2-methyl-1-naphthol,4-amino-3-methyl-1-naphthol, 4-methoxy-1-naphthol, 3-methoxy-2-naphthol, 5-methoxy-1-naphthol, 4-chloro-1-naphthol,1-chloro-2-naphthol, 1-bromo-2-naphthol, and 1-naphthol-4-sulfonic acid.

Preferably, naphthol derivatives of structures (IV) and (V) are obtained by a Mannich reaction wherein 1-naphthol (α-naphthol) or 2-naphthol (β-naphthol) is reacted with an aldehyde and an amine to form a Mannich base. Preferably, in one embodiment the mole ratio of amine to naphthol is within the range of from 1:1 to 1:3. Preferably, in another embodiment, the mole ratio of amine to naphthol is within the range of from 1:1 to 1:2. Preferably, in one embodiment the mole ratio of amine to aldehyde is within the range of from 1:1 to 1:6. Preferably, in another embodiment, the mole ratio of naphthol to aldehyde is within the range of from 1:1 to 1:3.

Preferably, the reaction is carried out in a one-step process by mixing the naphthol with the amine and treating this mixture with the aldehyde at the desired reaction temperature. Alternately, the aldehyde may be mixed with the amine and treated with the naphthol at the reaction temperature. Preferably, the reaction may be carried out at 40° C.-150° C. Preferably, in another embodiment, the reaction may be carried out at 80° C.-120° C. The product is obtained by distillation of water after the reaction is completed.

Preferably, the aldehyde compound used is represented by the structural formula RCOH, wherein R═H, C₁-C₁₀ alkyl, Ph, C₅-C₆ cycloaliphatic group or mixtures thereof. Suitable aldehydes are formaldehyde, acetaldehyde, propionaldehyde, butyraldehyde, pentanal, hexanal, octanal, heptanal, decanal, benzaldehyde, cyclopentanecarboxaldehyde, cyclohexanecarboxaldehyde. Preferred aldehydes are formaldehyde and acetaldehyde. Preferably, formaldehyde can be used as an aqueous solution or in the polymeric form, paraformaldehyde.

Preferably, the amine compound used in the reaction with 1-naphthol or 2-naphthol may be an alkylene polyamine e.g. ethylenediamine, a polyalkylene polyamine e.g. diethylenetriamine, triethylenetetramine, tetraethylenepentamine, N¹-(3-dimethylaminopropyl)propylenediamine (DMAPAPA), dimethylaminopropylamine (DMAPA), or an arylalkyl polyamine such as m-xylenediamine or a cycloaliphatic polyamine such as 4,4′-methylene-dicyclohexylamine (PACM) or a polyetherpolyamine such as Jeffamine D230.

In another embodiment, the curing agent composition further comprises another epoxy accelerator in addition to the naphthol or naphthol derivative. Preferably, in this further embodiment, the curing agent composition further comprises at least one compound selected from the group consisting of a boron trifluoride amine complex, a substituted phenol such as 2,4,6-tri(dimethylaminomethyl)phenol, a tertiary amine such as benzyldimethylamine or an imidazole, calcium nitrate, a carboxylic acid, salicylic acid, and sulfuric acid.

The present disclosure also provides for the use of a curing agent composition comprising a naphthol or naphthol derivative represented by the structures (I)-(V) and at least one polyamine having three or more active amine hydrogens as a hardener for epoxy resins.

The present disclosure is also directed to a method for producing a composition comprising the steps of (a) dissolving at least one naphthol or naphthol derivative in at least one polyamine to form a mixture; and (b) reacting the mixture with an epoxy resin component. Preferably, the at least one naphthol or naphthol derivative is represented by structure (1). Preferably, the at least one naphthol or naphthol derivative is represented by structures (II)-(V). Preferably, the at least one naphthol is selected from the group consisting of 4-methyl-1-naphthol, 2-methyl-1-naphthol,4-amino-3-methyl-1-naphthol, 4-methoxy-1-naphthol, 3-methoxy-2-naphthol, 5-methoxy-1-naphthol, 4-chloro-1-naphthol,1-chloro-2-naphthol, 1-bromo-2-naphthol, and 1-naphthol-4-sulfonic acid. Preferably, the at least one naphthol derivative is obtained by a Mannich reaction wherein 1-naphthol or 2-naphthol is reacted with an aldehyde and an amine to form a Mannich base.

The present disclosure is also directed to a method for producing a composition comprising the steps of (a) dissolving at least one naphthol or naphthol derivative in an epoxy resin component to form a mixture; and (b) reacting the mixture with at least one polyamine. Preferably, the at least one naphthol or naphthol derivative is represented by structure (1). Preferably, the at least one naphthol or naphthol derivative is represented by structures (II)-(V). Preferably, the at least one naphthol is selected from the group consisting of 4-methyl-1-naphthol, 2-methyl-1-naphthol,4-amino-3-methyl-1-naphthol, 4-methoxy-1-naphthol, 3-methoxy-2-naphthol, 5-methoxy-1-naphthol, 4-chloro-1-naphthol,1-chloro-2-naphthol, 1-bromo-2-naphthol, and 1-naphthol-4-sulfonic acid. Preferably, the at least one naphthol derivative is obtained by a Mannich reaction wherein 1-naphthol or 2-naphthol is reacted with an aldehyde and an amine to form a Mannich base.

The present disclosure also provides amine-epoxy compositions and the cured products produced therefrom. Another aspect of the invention relates to a composition comprising the reaction product of:
(a) at least one naphthol or naphthol derivative represented by the structure (1) below:

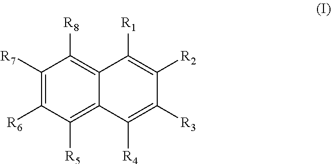

wherein R₁ and R₂ independently of each other=OH, H, C₁-C₁₀ alkyl, C₁-C₁₀ aryl, C₁-C₁₀ alkyl or aryl ether, NH₂, Cl, Br, I, NO₂, HSO₃, or X(CH₂)NHY with Y═C₁-C₁₀ alkyl, C₁-C₁₀ aryl, or a polyamine and X=Ph or C₁-C₄ alkyl; R₃-R₈═H, C₁-C₁₀ alkyl, C₁-C₁₀ aryl, C₁-C₁₀ alkyl or aryl ether, NH₂, Cl, Br, I, NO₂, or HSO₃; and wherein one of R₁ or R₂═OH;
(b) at least one polyamine having three or more active amine hydrogens; and
(c) an epoxy resin component comprising at least one multifunctional epoxy resin.

In a preferred embodiment, the composition comprises the reaction product of:
(a) at least one naphthol or naphthol derivative wherein the at least one naphthol or naphthol derivative is represented by the structures (II)-(V) below:

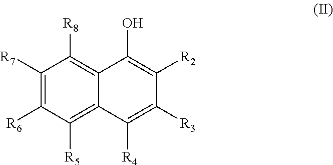

wherein R₂-R₈═H, C₁-C₁₀ alkyl, C₁-C₁₀ aryl, C₁-C₁₀ alkyl or aryl ether, NH₂, Cl, Br, I, NO₂, or HSO₃;

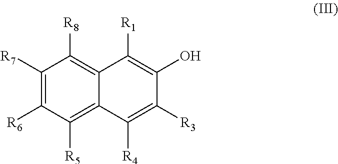

wherein R₁ and R₃-R₈═H, C₁-C₁₀ alkyl, C₁-C₁₀ aryl, C₁-C₁₀ alkyl or aryl ether, NH₂, Cl, Br, I, NO₂, or HSO₃;

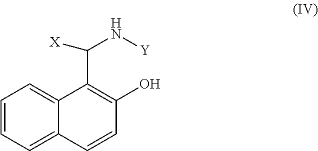

wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl; and

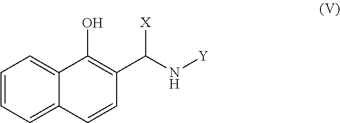

wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl; (b) at least one polyamine having three or more active amine hydrogens; and (c) an epoxy resin component comprising at least one multifunctional epoxy resin.

Preferred polyamines having three or more active amine hydrogens include diethylenetriamine (DETA), triethylenetetramine (TETA), tetraethylenepentamine (TEPA), pentaethylenehexamine (PEHA), hexamethylenediamine (HMDA),1,3-pentanediamine (DYTEK™ EP), 2-methyl-1,5-pentanediamine (DYTEK™A), triaminononane, N-(2-aminoethyl)-1, 3-propanediamine (N-3-Amine), N, N′-1, 2-ethanediylbis-1, 3-propanediamine (N₄-amine), or dipropylenetriamine; an arylaliphatic polyamine such as m-xylylenediamine (mXDA), or p-xylylenediamine; a cycloaliphatic polyamine such as 1,3-bis(aminomethyl)cyclohexylamine (1,3-BAC), isophorone diamine (IPDA), 4,4′-methylenebiscyclohexanamine,1,2-diaminocyclohexylamine (DCHA), aminopropylcyclohexylamine (APCHA), a methylene bridged poly (cycloaliphatic-aromatic) amine such as MPCA, an aromatic polyamine such as m-phenylenediamine, diaminodiphenylmethane (DDM), or diaminodiphenylsulfone (DDS); a heterocyclic polyamine such as N-aminoethylpiperazine (NAEP), or 3,9-bis(3-aminopropyl)2, 4,8, 10-tetraoxaspiro (5,5)undecane; a polyalkoxypolyamine where the alkoxy group can be an oxyethylene, oxypropylene, oxy-1, 2-butylene, oxy-1, 4-butylene or co-polymers thereof such as 4,7-dioxadecane-1, 10-diamine, I-propanamine, 3,3′-(oxybis (2, 1-ethanediyloxy))bis(diaminopropylated diethylene glycol ANCAMINE1922A),poly(oxy(methyl-1, 2-ethanediyl)), alpha-(2-aminomethylethyl)omega-(2-aminomethylethoxy) (JEFFAMINE D 230, D-400), triethyleneglycoldiamine and oligomers (JEFFAMINEXTJ-504, JEFFAMINE XTJ-512), poly(oxy(methyl-1, 2-ethanediyl)), alpha, alpha′-(oxydi-2, 1-ethanediyl)bis(omega-(aminomethylethoxy)) (JEFFAMINE XTJ-511), bis(3-aminopropyl)polytetrahydrofuran 350, bis(3-aminopropyl)polytetrahydro furan 750, poly(oxy(methyl-1, 2-ethanediyl)), α-hydro-ω-(2-aminomethylethoxy)ether with 2-ethyl-2-(hydroxymethyl)-1, 3-propanediol (3:1) (JEFFAMINE T-403), and diaminopropyldiaminopropyl dipropylene glycol.

Other preferred polyamine co-curing agents include amidoamines and polyamides. Polyamides are comprised of the reaction products of dimerized fatty acid (dimer acid) and polyethyleneamines, and usually a certain amount of monomeric fatty acid which helps to control molecular weight and viscosity. “Dimerized” or “dimer” or “polymerized” fatty acid refers to polymerized acids obtained from unsaturated fatty acids. They are described more fully in T. E. Breuer, ‘Dimer Acids’, in J. I. Kroschwitz (ed.), Kirk-Othmer Encyclopedia of Chemical Technology, 4′ Ed., Wiley, New York, 1993, Vol. 8, pp. 223-237. Common mono-functional unsaturated C-6 to C-20 fatty acids also employed in making polyamides include tall oil fatty acid (TOFA) or soya fatty acid or the like.

Further preferred polyamine co-curing agents include phenalkamines and Mannich bases of phenolic compounds with polyamines and formaldehyde.

Preferably, in one embodiment the weight ratio of the naphthol or naphthol-derived Mannich base and polyamine co-curing agent is 1:1 to 1:0.05. In another preferred embodiment, the weight ratio of the naphthol or naphthol-derived Mannich base and polyamine co-curing agent is 1:0.75 to 1:0.25.

Preferably, in one embodiment the amine-epoxy compositions of the present disclosure have stoichiometric ratios of epoxy groups in the epoxy resin component to amine hydrogens in the curing agent composition ranging from 1.5:1 to 0.7:1. Preferably, in one embodiment, such amine-epoxy compositions may have stoichiometric ratios of 1.5:1, 1.4:1, 1.3:1, 1.2:1, 1.1:1, 1:1, 0.9:1, 0.8:1, or 0.7:1. In another preferred embodiment, the stoichiometric ratio ranges from 1.3:1 to 0.7:1, or from 1.2:1 to 0.8:1, or from 1.1:1 to 0.9:1.

Preferably, in one embodiment curing agent compositions of the present disclosure have an amine hydrogen equivalent weight (AHEW) based on 100% solids from 50 to 500. The present disclosure, in another aspect, provides amine-epoxy compositions and the cured products produced therefrom. For example, an amine-epoxy composition, in accordance with the present disclosure, comprises the reaction product of a curing agent composition containing the novel compositions comprising at least one naphthol or naphthol derivative and having at least two active amine hydrogen atoms and epoxy resin component comprising at least one multifunctional epoxy resin.

Preferably, in one embodiment the naphthol or naphthol derivative is 0.5-50 wt. % relative to the amine in the curing agent composition. In a preferred embodiment, 5-30 wt. % relative to the amine may be used. In another preferred embodiment, the ratio of naphthol or naphthol derivative relative to the amine is 10-30 wt. %.

Preferred naphthol compounds are naphthol Mannich bases, 1-naphthol (α-naphthol) and 2-naphthol (β-naphthol).

The present disclosure also includes use of a curing agent as described above, together with at least one epoxy resin component, for the preparation of hardened articles of manufacture. Preferably, such articles may include, but are not limited to a coating, a primer, a sealant, a curing compound, a construction product, a flooring product, a composite product, laminate, potting compounds, grouts, fillers, cementitious grouts, or self-leveling flooring. Additional components or additives may be used together with the compositions of the present disclosure to produce articles of manufacture. Further, such coatings, primers, sealants, curing compounds or grouts may be applied to metal or cementitious substrates. Preferably, the article is a coating. Preferably, in one embodiment the coating is a flexible epoxy coating. Preferably, in one embodiment the coating is prepared at ambient temperature. Preferably, in another embodiment the coating is prepared at a sub-ambient temperature as low as 0° C.

The encapsulation of an epoxy curing agent with a solid naphthol e.g. 2-naphthol also provides a useful technique for one component (1K) epoxy curing agents. Such application is known for phenol-formaldehyde resins in combination with epoxy amine curing agents. The encapsulation of amines with solid naphthol compounds allows for a faster/lower temperature curing 1K epoxy system.

The relative amount chosen for the epoxy resin component versus that of the curing agent composition, may vary depending upon, for example, the end-use article, its desired properties, and the fabrication method and conditions used to produce the end-use article. For instance, in coating applications using certain amine-epoxy compositions, incorporating more epoxy resin relative to the amount of the curing agent composition may result in coatings which have increased drying time, but with increased hardness and improved appearance as measured by gloss. Amine-epoxy compositions of the present disclosure have stoichiometric ratios of epoxy groups in the epoxy resin component to amine hydrogens in the curing agent composition ranging from 1.5:1 to 0.7:1. For example, such amine-epoxy compositions may have stoichiometric ratios of 1.5:1, 1.4:1, 1.3:1, 1.2:1, 1.1:1, 1:1, 0.9:1, 0.8:1, or 0.7:1. In another aspect, the stoichiometric ratio ranges from 1.3:1 to 0.7:1, or from 1.2:1 to 0.8:1, or from 1.1:1 to 0.9:1.

Amine-epoxy compositions of the present disclosure comprise the reaction product of a curing agent composition and an epoxy resin component comprising at least one multifunctional epoxy resin. Multifunctional epoxy resin, as used herein, describes compounds containing 2 or more 1,2-epoxy groups per molecule. Preferably, the epoxy resin component is selected from the group consisting of aromatic epoxy resin, alicyclic epoxy resin, aliphatic epoxy resin, glycidyl ester resin, thioglycidyl ether resin, N-glycidyl ether resin, and combinations thereof.

Preferable aromatic epoxy resin suitable for use in the present disclosure comprises the glycidyl ethers of polyhydric phenols, including the glycidyl ethers of dihydric phenols. Further preferred are the glycidyl ethers of resorcinol, hydroquinone, bis-(4-hydroxy-3,5-difluorophenyl)-methane, 1,1-bis-(4-hydroxyphenyl)-ethane, 2,2-bis-(4-hydroxy-3-methylphenyl)-propane, 2,2-bis-(4-hydroxy-3,5-dichlorophenyl) propane, 2,2-bis-(4-hydroxyphenyl)-propane (commercially known as bisphenol A), bis-(4-hydroxyphenyl)-methane (commercially known as bisphenol F, and which may contain varying amounts of 2-hydroxyphenyl isomers), and the like, or any combination thereof. Additionally, advanced dihydric phenols of the following structure also are useful in the present disclosure:

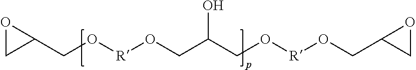

wherein R′ is a divalent hydrocarbon radical of a dihydric phenol, such as those dihydric phenols listed above, and p is an average value between 0 and 7. Materials according to this formula may be prepared by polymerizing mixtures of a dihydric phenol and epichlorohydrin, or by advancing a mixture of a diglycidyl ether of the dihydric phenol and the dihydric phenol. While in any given molecule the value of p is an integer, the materials are invariably mixtures which may be characterized by an average value of p which is not necessarily a whole number. Polymeric materials with an average value of p between 0 and 7 may be used in one aspect of the present disclosure.

In one aspect of the present disclosure, the at least one multifunctional epoxy resin is preferably a diglycidyl ether of bisphenol-A (DGEBA), an advanced or higher molecular weight version of DGEBA, a diglycidyl ether of bisphenol-F, a diglycidyl ether of novolac resin, or any combination thereof. Higher molecular weight versions or derivatives of DGEBA are prepared by the advancement process, where excess DGEBA is reacted with bisphenol-A to yield epoxy terminated products. The epoxy equivalent weights (EEW) for such products range from 450 to 3000 or more. Because these products are solid at room temperature, they are often referred to as solid epoxy resins.

In preferred embodiments, the at least one multifunctional epoxy resin is the diglycidyl ether of bisphenol-F or bisphenol-A represented by the following structure:

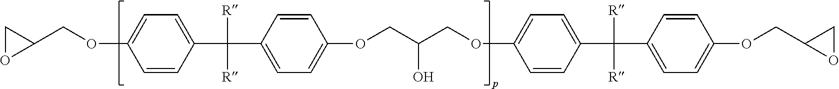

wherein R″=H or CH₃, and p is an average value between 0 and 7. DGEBA is represented by the above structure when R″=CH₃ and p=0. DGEBA or advanced DGEBA resins are often used in coating formulations due to a combination of their low cost and high performance properties. Commercial grades of DGEBA having an EEW ranging from 174 to 250, and more commonly from 185 to 195, are readily available. At these low molecular weights, the epoxy resins are liquids and are often referred to as liquid epoxy resins. It is understood by those skilled in the art that most grades of liquid epoxy resin are slightly polymeric, since pure DGEBA has an EEW of 174. Resins with EEWs between 250 and 450, also prepared by the advancement process, are referred to as semi-solid epoxy resins because they are a mixture of solid and liquid at room temperature. Multifunctional resins with EEWs based on solids of 160 to 750 are useful in the present disclosure. In another aspect the multifunctional epoxy resin has an EEW in a range from 170 to 250.

Examples of alicyclic epoxy compounds include, but are not limited to, polyglycidyl ethers of polyols having at least one alicyclic ring, or compounds including cyclohexene oxide or cyclopentene oxide obtained by epoxidizing compounds including a cyclohexene ring or cyclopentene ring with an oxidizer. Some particular examples include, but are not limited to hydrogenated bisphenol A diglycidyl ether; 3,4-epoxycyclohexylmethyl-3,4-epoxycyclohexyl carboxylate; 3,4-epoxy-1-methylcyclohexyl-3,4-epoxy-1-methylhexane carboxylate; 6-methyl-3,4-epoxycyclohexylmethyl-6-methyl-3,4-epoxycyclohexane carboxylate; 3,4-epoxy-3-methylcyclohexylmethyl-3,4-epoxy-3-methylcyclohexane carboxylate; 3,4-epoxy-5-methylcyclohexylmethyl-3,4-epoxy-5-methylcyclohexane carboxylate; bis(3,4-epoxycyclohexylmethyl)adipate; methylene-bis(3,4-epoxycyclohexane); 2,2-bis(3,4-epoxycyclohexyl)propane; dicyclopentadiene diepoxide; ethylene-bis(3,4-epoxycyclohexane carboxylate); dioctyl epoxyhexahydrophthalate; and di-2-ethylhexyl epoxyhexahydrophthalate.

Examples of aliphatic epoxy compounds include, but are not limited to, polyglycidyl ethers of aliphatic polyols or alkylene-oxide adducts thereof, polyglycidyl esters of aliphatic long-chain polybasic acids, homopolymers synthesized by vinyl-polymerizing glycidyl acrylate or glycidyl methacrylate, and copolymers synthesized by vinyl-polymerizing glycidyl acrylate or glycidyl methacrylate and other vinyl monomers. Some particular examples include, but are not limited to, glycidyl ethers of polyols, such as 1,4-butanediol diglycidyl ether; 1,6-hexanediol diglycidyl ether; a triglycidyl ether of glycerin; a triglycidyl ether of trimethylol propane; a tetraglycidyl ether of sorbitol; a hexaglycidyl ether of dipentaerythritol; a diglycidyl ether of polyethylene glycol; and a diglycidyl ether of polypropylene glycol; polyglycidyl ethers of polyether polyols obtained by adding one type, or two or more types, of alkylene oxide to aliphatic polyols, such as ethylene glycol, propylene glycol, trimethylol propane, and glycerin.

Glycidyl ester resins are obtained by reacting a polycarboxylic acid compound having at least two carboxyl acid groups in the molecule and epichlorohydrin. Examples of such polycarboxylic acids include aliphatic, cycloaliphatic, and aromatic polycarboxylic acids. Examples of aliphatic polycarboxylic acids include oxalic acid, succinic acid, glutaric acid, adipic acid, pimelic acid, sebacic acid, suberic acid, azelaic acid, or dimerised or trimerised linoleic acid. Cycloaliphatic polycarboxylic acids include tetrahydrophthalic acid, 4-methyltetrahydrophthalic acid, hexahydrophthalic acid or 4-methylhexahydrophthalic acid and aromatic polycarboxylic acids include phthalic acid, isophthalic acid or terephthalic acid.

Thioglycidyl ether resins are derived from dithiols, for example, ethane-1,2-dithiol or bis(4-mercaptomethylphenyl) ether.

N-glycidyl resins are obtained by dehydrochlorination of the reaction products of epichlorohydrin with amines containing at least two amine hydrogen atoms. Such amines are, for example, aniline, n-butylamine, bis(4-aminophenyl)methane, m-xylylenediamine or bis(4-methylaminophenyl)methane. The N-glycidyl resins also include, however, triglycidyl isocyanurate, N,N′-diglycidyl derivatives of cycloalkylene ureas, e.g., ethylene urea or 1,3-propylene urea, and diglycidyl derivatives of hydantoins, e.g., 5,5-dimethylhydantoin.

For one or more of the embodiments, the resin component further includes a reactive diluent. Reactive diluents are compounds that participate in a chemical reaction with the hardener component during the curing process and become incorporated into the cured composition and are monofunctional epoxides. Reactive diluents may also be used to vary the viscosity and/or cure properties of the curable compositions for various applications. For some applications, reactive diluents may impart a lower viscosity to influence flow properties, extend pot life and/or improve adhesion properties of the curable compositions. For example, the viscosity may be reduced to allow an increase in the level of pigment in a formulation or composition while still permitting easy application, or to allow the use of a higher molecular weight epoxy resin. Thus, it is within the scope of the present disclosure for the epoxy component, which comprises at least one multifunctional epoxy resin, to further comprise a monofunctional epoxide. Examples of monoepoxides include, but are not limited to, styrene oxide, cyclohexene oxide and the glycidyl ethers of phenol, cresols, tert-butylphenol, other alkyl phenols, butanol, 2-ethylhexanol, C₄ to C₁₄ alcohols, and the like, or combinations thereof. The multifunctional epoxy resin may also be present in a solution or emulsion, with the diluent being water, an organic solvent, or a mixture thereof. The amount of multifunctional epoxy resin may range from 50% to 100%, 50% to 90%, 60% to 90%, 70% to 90%, and in some cases 80% to 90%, by weight, of the epoxy component. For one or more of the embodiments, the reactive diluent is less than 60 weight percent of a total weight of the resin component.

Particularly suitable multifunctional epoxy compounds are the diglycidyl ethers of bisphenol-A and bisphenol-F, the advanced diglycidyl ethers of bisphenol-A and bisphenol-F, and the epoxy novolac resins. The epoxy resin may be a single resin, or it may be a mixture of mutually compatible epoxy resins.

Compositions of the present disclosure may be used to produce various articles of manufacture. Depending on the requirements during the manufacturing of or for the end-use application of the article, various additives may be employed in the formulations and compositions to tailor specific properties. These additives include, but are not limited to, solvents (including water), accelerators, plasticizers, fillers, fibers, such as glass or carbon fibers, pigments, pigment dispersing agents, rheology modifiers, thixotropes, flow or leveling aids, surfactants, defoamers, biocides, or any combination thereof. It is understood that other mixtures or materials that are known in the art may be included in the compositions or formulations and are within the scope of the present disclosure.

The present disclosure also is directed to articles of manufacture comprising the compositions disclosed herein. For example, an article may comprise an amine-epoxy composition which comprises the reaction product of a curing agent composition and an epoxy composition. The curing agent composition may comprise the naphthol or naphthol derived Mannich base. The epoxy resin component may comprise at least one multifunctional epoxy resin. Optionally, various additives may be present in the compositions or formulations used to produce fabricated articles, dependent upon the desired properties. These additives may include, but are not limited to, solvents (including water), accelerators, plasticizers, fillers, fibers, such as glass or carbon fibers, pigments, pigment dispersing agents, rheology modifiers, thixotropes, flow or leveling aids, surfactants, defoamers, biocides, or any combination thereof. The selection and amount of these additives is at the option of the formulator.

In another embodiment, the naphthol or naphthol derived Mannich base accelerator curing composition may be combined with other epoxy cure accelerators. Preferably, representative accelerators which may be used include: boron trifluoride amine complexes, substituted phenols such as 2,4,6-tri(dimethylaminomethyl)phenol, tertiary amines such as benzyldimethylamine and imidazoles. Calcium nitrate, carboxylic acids, salicylic acid, sulfuric acid etc.

Articles in accordance with the present disclosure include, but are not limited to, a coating, a primer, a sealant, a curing compound, a construction product, a flooring product, a composite product, laminate, potting compounds, grouts, fillers, cementitious grouts, or self-leveling flooring. Coatings based on these amine-epoxy compositions may contain diluents, such as water or organic solvents, as needed for the particular application. Coatings may contain various types and levels of pigments for use in paint and primer applications. Amine-epoxy coating compositions comprise a layer having a thickness ranging from 40 to 400 μm (micrometer), preferably 80 to 300 μm, more preferably 100 to 250 μm, for use in a protective coating applied onto metal substrates. In addition, for use in a flooring product or a construction product, coating compositions comprise a layer having a thickness ranging from 50 to 10,000 μm, depending on the type of product and the required end-properties. A coating product that delivers limited mechanical and chemical resistances comprises a layer having a thickness ranging from 50 to 500 μm, preferably 100 to 300 μm; whereas a coating product, such as, for example, a self-leveling floor that delivers high mechanical and chemical resistances comprises a layer having a thickness ranging from 1,000 to 10,000 μm, preferably 1,500 to 5,000 μm.

Various substrates are suitable for the application of coatings of this invention with proper surface preparation, as is well known to one of ordinary skill in the art. Such substrates include, but are not limited to, concrete and various types of metals and alloys, such as steel and aluminum. Coatings of the present disclosure are suitable for the painting or coating of large metal objects or cementitious substrates including ships, bridges, industrial plants and equipment, and floors.

Coatings of this invention may be applied by any number of techniques including spray, brush, roller, paint mitt, and the like. In order to apply very high solids content or 100% solids coatings of this invention, plural component spray application equipment may be used, in which the amine and epoxy components are mixed in the lines leading to the spray gun, in the spray gun itself, or by mixing the two components together as they leave the spray gun. Using this technique may alleviate limitations with regard to the pot life of the formulation, which typically decreases as both the amine reactivity and the solids content increases. Heated plural component equipment may be employed to reduce the viscosity of the components, thereby improving ease of application.

Construction and flooring applications include compositions comprising the amine-epoxy compositions of the present disclosure in combination with concrete or other materials commonly used in the construction industry. Applications of compositions of the present disclosure include, but are not limited to, its use as a primer, a deep penetrating primer, a coating, a curing compound, and/or a sealant for new or old concrete, such as referenced in ASTM C309-97, which is incorporated herein by reference. As a primer or a sealant, the amine-epoxy compositions of the present disclosure may be applied to surfaces to improve adhesive bonding prior to the application of a coating. As it pertains to concrete and cementitious application, a coating is an agent used for application on a surface to create a protective or decorative layer or a coat. Crack injection and crack filling products also may be prepared from the compositions disclosed herein. Amine-epoxy compositions of the present disclosure may be mixed with cementitious materials, such as concrete mix, to form polymer or modified cements, tile grouts, and the like. Non-limiting examples of composite products or articles comprising amine-epoxy compositions disclosed herein include tennis rackets, skis, bike frames, airplane wings, glass fiber reinforced composites, and other molded products.

In a particular use of the curing agent composition of the present disclosure, coatings may be applied to various substrates, such as concrete and metal surfaces at low temperature, with fast cure speed and good coating appearance. This is especially important for top-coat application where good aesthetics is desired and provides a solution to a long-standing challenge in the industry where fast low-temperature cure with good coating appearance remains to be overcome. With fast low-temperature cure speed, the time service or equipment is down may be shortened, or for outdoor applications, the work season may be extended in cold climates.

Fast epoxy curing agents enable amine-cured epoxy coatings to cure in a short period of time with a high degree of cure. The cure speed of a coating is monitored by thin film set time (TFST) which measures the time period a coating dries. The thin film set time is categorized in 4 stages: phase 1, set to touch; phase 2, tack free: phase 3, dry hard; and phase 4, dry through. The phase 3 dry time is indicative of how fast a coating cures and dries. For a fast ambient cure coating, phase 3 dry time is less than 4 hours, or less than 3 hours, or preferred to be less than 2 hours. Low temperature or sub-ambient temperature cure typically refers to cure temperature below ambient temperature, 10° C. or 5° C., or 0° C. in some cases. For a fast low temperature cure, phase 3 dry time at 5° C. is less than 6 hours, with a significant productivity benefit being provided for values where phase 3 dry times are less than 4 hours and preferably less than 3 hours.

How well a coating cures is measured by the degree of cure. Degree of cure is often determined by using DSC (differential scanning calorimetry) technique which is well-known to those skilled in the art. A coating that cures thoroughly will have a degree of cure at ambient temperature (25° C.) of at least 85%, or at least 90%, or at least 95% after 7 days. A coating that cures thoroughly will have a degree of cure at 5° C. of at least 80%, or at least 85%, or at least 90% after 7 days.

Many of the fast low temperature epoxy curing agents may cure an epoxy resin fast. However due to poor compatibility of the epoxy resin and curing agents especially at low temperature of 10 degrees Celsius or 5 degrees Celsius, there is phase separation between resin and curing agent and curing agent migrating to coating surface, resulting in poor coating appearance manifested as sticky and cloudy coatings. Good compatibility between epoxy resin and curing agent leads to clear glossy coating with good carbamation resistance and good coating appearance. The curing agent compositions of the present disclosure offers the combination of fast cure speed, good compatibility and high degree of cure.

### EXAMPLES

The various aspects of this invention can be used alone or in combination. Certain aspects of the invention are illustrated by the following Examples. These Examples shall not limit the scope of the appended claims.

### Example 1

### Synthesis of 2-Naphthol Mannich Base from Formaldehyde and Triethylenetetramine (TETA)

A 3-neck 1 L round bottom flask equipped with N₂ inlet, addition funnel and temperature probe was charged with 2-naphthol (1.0 mole) and triethylentetramine (g, 1.0 mole). The mixture was heated to 80° C. A 37% solution of formaldehyde (81 g, 37 wt. %, 30 g, 1.0 mole) was added to maintain a reaction temperature of 80-90° C. After the addition, the mixture was kept at 90-95° C. for 1 h. Water was distilled at 120° C. and the product was obtained as a light brown liquid. This product was cooled to ambient temperature and tested for epoxy cure performance.

### Performance Testing

Curing agent mixtures were prepared by mixing the components given in the above examples. with the epoxy component of standard bisphenol-A based epoxy resin of (Epon 828, DER 331 type), EEW 190, unless specified otherwise. They were then mixed employing a stoichiometric level of 1:1 (amine: epoxy equivalents).

### Thin Film Set Time

The dry time or thin film set time (TFST) was determined using a Beck-Koller recorder, in accordance with ASTM D5895. The amine-epoxy coatings were prepared on standard glass panels at a wet film thickness of 150 μm WFT (wet film thickness) using a Bird applicator resulting in dry film thicknesses of ±100 μm. The coatings were cured at 23° C. and 5° C. and 60% relative humidity (RH) in a Lunaire (TPS) environmental chamber.

### Differential Scanning Calorimetric Study

Thermal study was conducted using DSC to understand the cure kinetics, reactivity and Tg on 1-2 mg of sample.

### Viscosity Cure Profile

Latency study was determined using Brookfield viscometer with Wingather software to generate viscosity cure profile.

TABLE 1
Summary of the cure performance of the TETA/2-naphthol Mannich base

| | | Viscosity | | | | |
|---|---|---|---|---|---|---|
| 2- | | cure | | DSC % | DSC % | TFST |
| Naphthol/TETA | Viscosity | Time to | DSC | cure, | cure, | 23° C./ |
| Mannich base | (cP) | double | DH, Tg | 25° C. | 5° C. | 5° C. |
| phr | 25° C. | (min) | ° C. | 1 d/2 d/7 d | 1 d/2 d/7 d | (h) |
| 50 | 7667; | <4 | ND | ND | ND | ND |
| | 80 wt. % | | | | | |
| | in benzyl | | | | | |
| | alcohol | | | | | |
| 25 | 7667; | 21 | 267/85 | 77/78/89 | 55/60/74 | 3/6 |
| | 80 wt. % | | | | | glossy |
| | in benzyl | | | | | |
| | alcohol | | | | | |
| 18 | 7667; | 19 | 189/60 | 81/84/89 | 39/63/72 | 5/9 |
| | 80 wt. % | | | | | glossy |
| | in benzyl | | | | | |
| | alcohol | | | | | |
| 10 | 7667; | 29 | 141/24 | 84/84/92 | 64/71/83 | >12/>24 |
| | 80 wt. % | | | | | glossy |
| | in benzyl | | | | | |
| | alcohol | | | | | |

### Example 2

### Control Curing Agent: A1—Control

In a 3-neck round bottom flask equipped with Nitrogen inlet, addition funnel and temperature probe was charged with 50 Grams of Nonyl Phenol and 50 grams of 2-aminomethylpiperazine. The mixture was heated to 40 C and stirred until a homogenous mixture was obtained.

### Formulated Curing Agent: A1—Exptl

A 3-neck ½L round bottom flask equipped with N₂ inlet, addition funnel and temperature probe was charged with 50 grams of 2-naphthol and Aminoethylpiperazine 50 Grams. The mixture was heated to 40 C and stirred until the 2-naphthol dissolved in Amintoethyl piperazine. The comparative performance properties of Control Curing Agent A-1-Control and the formulated curing agent A-1 Exptl are given in Table 2.

### Example 3

### Control Curing Agent—A2—Control

In a 3 neck round bottom flask equipped with Nitrogen inlet, additional funnel and temperature probe was charged with 26.3 grams of M-xylenediamine, 5 grams of Nonyl Phenol and 25.3 grams of Trimethyl hexamethylenediamine and 43.4 grams of Paratertiary butyl phenol. The mixture was heated and stirred until the para tertiary butyl phenol is completely dissolved.

### Formulated Curing Agent—A2—Exptl

A 3-neck ½L round bottom flask equipped with N₂ inlet, addition funnel and temperature probe were charged with 35 grams of 2-naphthol and Isophorone diamine and 25.3 Grams of Trimethyl hexamethylenediamine. The mixture was heated to 40 C and stirred until the 2-naphthol is completely dissolved in the amine mixture. The comparative performance properties of Control Curing Agent A-2Control and the formulated curing agent A-2 Exptl are given in Table 2.

### Example 4

### Control Curing Agent—A2—Comparative

In a 3 neck round bottom flask equipped with Nitrogen inlet, additional funnel and temperature probe was charged with 26.3 grams of M-xylenediamine, 5 grams of Nonyl Phenol and 25.3 grams of Trimethyl hexamethylenediamine and 43.4 grams of Paratertiary butyl phenol. The mixture was heated and stirred until the para tertiary butyl phenol is completely dissolved.

### Formulated Curing Agent—A3—Exptl

A 3-neck ½L round bottom flask equipped with N₂ inlet, addition funnel and temperature probe were charged with 43.4 grams of 2-naphthol (0.34 moles) and m-xylene diamine (0.23 mole) and 25.3 Grams of Trimethyl hexamethylenediamine (0.16 moles). The mixture was heated to 40 C and stirred until the 2-naphthol is completely dissolved in the amine mixture. The comparative performance properties of Control Curing Agent A-2Control and the formulated curing agent A-3 Exptl are given in Table 2.

### Example 5

### Formulated Curing Agent—A4—Exptl

A 3-neck ½L round bottom flask equipped with N₂ inlet, addition funnel and temperature probe was charged with 65 grams of Phenalkamine curing agent Sunmide CX-1151 commercially available from Evonik Corporation and 35 grams of grams of 2-naphthol was added and heated to 40 C and stirred until the 2-naphthol is completely dissolved in the phenalkamine curing agent. The comparative performance properties of formulated curing agent in example 5 is compared to Sunmide CX1151 curing agent is given in Table 3.

### Example 6

### Formulated Curing Agent—A5—Exptl

A 3-neck ½L round bottom flask equipped with N₂ inlet, addition funnel and temperature probe was charged with 65 grams of Triethylenetetramine based polyamide curing agent Ancamide350A commercially available from Evonik Corporation and 33.4 grams of grams of 2-naphthol was added and heated to 40 C and stirred until the 2-naphthol is completely dissolved in the polyamide. The performance properties of the formulated cuing agent A5 was compared to Ancamide350A and given in Table 3.

### Example 7

### Formulated Curing Agent—A6—Exptl

A 3-neck ½L round bottom flask equipped with N₂ inlet, addition funnel and temperature probe was charged with 65 grams of Sunmide CX105 a commercially available ethylenediamine based Phenalkamine curing agent commercially available from Evonik Corporation and 30 grams of 2-naphthol) was added and heated to 40 C and stirred until the 2-naphthol is completely dissolved in the phenalkamine. The performance properties of formulated curing agent A-6-Exptl was compared to Sunmide CX105 Phenalkamine curing agent and given in Table 3.

### Example 8

### Formulated Curing Agent—A7—Exptl

A 3-neck ½L round bottom flask equipped with N₂ inlet, addition funnel and temperature probe was charged with 70 grams of Ancamine 2280 Cycloaliphatic curing agent commercially available from Evonik Corporation and 30 grams of composition 040-141 (A2) was added and heated to 40 C and stirred until the 2-naphthol is completely dissolved in the cycloaliphatic curing agent. The performance properties of Formulated curing agent A-7 in example 8 was compared to Ancamine A2280 and given in Table 3.

### Example Application and Performance Testing

### Performance Testing

Curing agent mixtures were prepared by mixing the components given in the above examples with the epoxy component of standard bisphenol-A based epoxy resin of (Epon 828, DER 331 type), EEW 190, unless specified otherwise. They were then mixed employing a stoichiometric level of 1:1 (amine: epoxy equivalents).

The following application testing was conducted on Formulated Curing agent examples A1-A9 and results are provided in Tables 2 and 3.
Thin film set times—ASTM D 5895
Gel time—ASTM D 2471
Persoz Hardness—ASTM D 4366
Shore D hardness ASTM—D2240

TABLE 2
Physical and test Properties

| | A1 - | A1 - | A2- | A2 | A2- | A3- |
|---|---|---|---|---|---|---|
| Curing Agent | Control | Exptl | Control | Exptl | Control | Exptl |
| Gel Time (150 gram mix) | 7 | 3 | 12 | 10 | 12 | 10 |
| 25° C. | | | | | | |
| MEK Double rubs RT 7 | 112 | 200 | 108 | 160 | 108 | 200 |
| Day Cure | | | | | | |
| WFT (mils) | 3 | 3 | 3 | 3 | 3 | 3 |
| Substrate | B952 | B952 | B952 | B952 | B952 | B952 |
| TEST I/II/III @ 25° C. | .5/1/1.5 | .25/.5/.75 | 1/1.75/2.25 | 75/1.5/2 | 1/1.75/2.25 | .75/1.5/1.75 |
| TEST I/II/III @5° C. | .75/2/2.5 | .5/.75/1 | 1/2.25/3 | 1/2/2.5 | 1/2.25/3 | .75/2/2.25 |
| WFT (mils) | 3 | 3 | 3 | 3 | 3 | 3 |
| Relative Humidity | 50% | 50% | 50% | 50% | 50% | 50% |
| Substrate | Glass | Glass | Glass | Glass | Glass | Glass |
| Persoz hardness (25° C.) | 371 | 370 | 383 | 387 | 383 | 388 |
| Day 7 | | | | | | |
| Persoz hardness (5° C.) | 266 | 321 | 314 | 320 | 314 | 335 |
| Day 7 | | | | | | |
| WFT (mils) | 3 | 3 | 3 | 3 | 3 | 3 |
| Relative Humidity | 50% | 50% | 50% | 50% | 50% | 50% |
| Substrate | B952 | B952 | B952 | B952 | B952 | B952 |
| Shore D (25° C. Cure) | 86/86/85 | 72/87/85 | 84/84/84 | 82/84/85 | 84/84/84 | 86/85/87 |

The formulated curing agents A-I Exptl to A-3-Exptl provides increased gel and thin film set times compared to controls. The formulated curing agents shows improved MEK double rub resistance which indicates the extent of reaction of formulated curing agents with epoxy resin is much higher compared to the controls. The Persoz hardness data also supports higher extent of reaction for the formulated curing agents.

TABLE 3
Physical and application properties

| | | Sunmide | | Sunmide | | Sunmide | | Ancamine | |
|---|---|---|---|---|---|---|---|---|---|
| | | CX-1151 | A4 | 350A | A5 | CX-105 | A6 | 2280 | A7 |
| Gel Time | | | | | | | | 50 | 21 |
| (150 gram | | | | | | | | | |
| mix) 25° C. | | | | | | | | | |
| MEK | <200 | 200 | 160 | 200 | 200 | | | | |
| Double | | | | | | | | | |
| rubs RT 7 | | | | | | | | | |
| Day Cure | | | | | | | | | |
| WFT (mils) | | 3 | 3 | 3 | 3 | | | | |
| Substrate | | B952 | B952 | B952 | B952 | | | | |
| TEST I/II/III | hrs | .5/1.25/1.75 | .25/.5/.75 | 5/8/9.5 | 1.75/4/4.75 | 4/6.5/7.5 | 0.75/1.5/2.5 | 2/3.75/6 | 1.25/2.5./3.75 |
| @ 25° C. | | | | | | | | | |
| TEST I/II/III | hrs | 1/3.5/4.5 | X/1.5/2.5 | 10/X/X | 4/9/15.5 | 6/18/24 | 1/4/6.5 | 3/8/14.5 | 3/6.5/10.5 |
| @5° C. | | | | | | | | | |
| WFT (mils) | | 3 | 3 | 3 | 3 | | | | |
| Relative | | 50% | 50% | 50% | 50% | | | | |
| Humidity | | | | | | | | | |
| Substrate | | Glass | Glass | Glass | Glass | | | | |
| Persoz | Secs | 280 | 111 | 282 | 275 | | | | |
| hardness | | | | | | | | | |
| (25° C.) | | | | | | | | | |
| Day 7 | | | | | | | | | |
| WFT (mils) | | 3 | 3 | 3 | 3 | | | | |
| Relative | | 50% | 50% | 50% | 50% | | | | |
| Humidity | | | | | | | | | |
| Substrate | | B952 | B952 | B952 | B952 | | | | |
| WFT (mils) | | 3 | 3 | 3 | 3 | | | | |
| Relative | | 50% | 50% | 50% | 50% | | | | |
| Humidity | | | | | | | | | |
| Direct | lb/inch | 92 | 112 | 80 | 4 | | | | |
| impact (7 | | | | | | | | | |
| Day) RT | | | | | | | | | |
| Reverse | lb/inch | 28 | 160 | 80 | X | | | | |
| Impact (7 | | | | | | | | | |
| Day) RT | | | | | | | | | |
| Direct | lb/inch | 100 | 152 | 68 | 160 | | | | |
| impact (7 | | | | | | | | | |
| Day) 5° C. | | | | | | | | | |
| Reverse | lb/inch | 84 | 156 | 104 | 160 | | | | |
| Impact (7 | | | | | | | | | |
| Day) 5° C. | | | | | | | | | |
| WFT (mils) | | 3 | 3 | 3 | 3 | | | | |
| Relative | | 50% | 50% | 50% | 50% | | | | |
| Humidity | | | | | | | | | |
| Substrate | | B963 | B955 | B964 | B958 | | | | |
| Shore D | 1/3/7 | 43/46/49 | 43/47/50 | 63/78/78 | 62/78/80 | | | | |
| (25° C. Cure) | Day | | | | | | | | |

For Experimental samples A4-A7 provide much fasters thin film set times compared to samples without the 2-naphthol.

### Example 9

The following examples describes the addition of 2-naphthol on the Epoxy resin side instead of adding it on the amine side.
Base resin preparation—(DER 354 Exptl) (Compare the compounds without naphthol)

A 3-neck ½L round bottom flask equipped with N₂ inlet, addition funnel and temperature probe were charged with 75 grams of Bisphenol F Diglycidyl Ether epoxy resin with an Epoxy equivalent weight of 228. 25 grams of 2-naphthol was added and heated to 40 C and stirred until the 2-naphthol is completely dissolved in the in the resin. The DER 354 exptl. resin described in this example is then added to DER 354 Bisphenol resin as shown in Table 1. The weight percent of total 2-napthol for Resin formulation numbers B1-B5 and the epoxy equivalent weights given in Table 4.

TABLE 4
Preparation with 2-naphthol in Epoxy resin

| | | Formulation | | | | |
|---|---|---|---|---|---|---|
| | | B1 | B2 | B3 | B4 | B5 |
| DER354 | 171 | 100 | 0 | 50 | 75 | 87.5 |
| DER354 | 228 | 0 | 100 | 50 | 25 | 12.5 |
| EXPTl | | | | | | |
| | | | | 100 | 100 | 100 |
| % 2- | | 100 | 25 | 12 | 6.5 | 3.125 |
| naphthol | | | | | | |
| EEW | | | 228 | 195 | 182 | 177 |

### Application and Performance Testing on Formulated Resins—Samples—B1-B5

The DER 354 (Exptl resin) was mixed with standard Bisphenol F diglycidyl ether epoxy resin as described in Table 4 and then cured with (1) a cycloaliphatic curing agent (Ancamine 2791), (2) an aliphatic epoxy curing agent (Ancamine 2739), and (3) a polyamide epoxy curing agent (Ancamide 2769). Thin film set times of DER 354 Exptl resin when cured with (1) a cycloaliphatic curing agent (A 2791), (2) an aliphatic curing agent (A 2739), and (3) a polyamide curing agent (A 2769) at both ambient and low temperature are shown below in Table 5.

TABLE 5
Thin Film Set Times
DER 354 Resin

| | | 25% | 12.5% | 6.25% | 3.125% | 0% |
|---|---|---|---|---|---|---|
| Ladder Study @ RT | | | | | | |
| Ancamine 2791 | I | 0.75 | 1.5 | 2.25 | 2.5 | 2.75 |
| | II | 1.5 | 2.5 | 3.25 | 4 | 4.5 |
| | III | 2.25 | 3.5 | 4.5 | 5.75 | 7.75 |
| Ancamine 2739 | I | 0.5 | 1.25 | 2.25 | 2.5 | 2.5 |
| | II | 1.5 | 2.5 | 3.5 | 4 | 4.5 |
| | III | 2.25 | 3.5 | 4.5 | 5 | 6 |
| Ancamide 2769 | I | 1.75 | 3.5 | 5 | 6 | 5.5 |
| | II | 4.25 | 7.75 | 9.5 | 11 | * |
| | III | 7.5 | 9.25 | 12 | 14 | * |
| Ladder Study @ 5 C. | | | | | | |
| Ancamine 2791 | I | 0.5 | 1 | 3.5 | 5 | 6 |
| | II | 3 | 4 | 8.5 | 11 | 13 |
| | III | 5 | 8 | 11 | 15.5 | 19.5 |
| Ancamine 2739 | I | 1.5 | 2 | 4 | 4 | 5 |
| | II | 4 | 5.5 | 8 | 9 | 10 |
| | III | 6 | 8.5 | 10.5 | 12 | 13.5 |
| Ancamide 2769 | I | 2.5 | 6 | 8 | 10 | 9 |
| | II | 9 | 14.5 | 15 | 18 | * |
| | III | 14.5 | 19.5 | * | * | * |

The addition of 2-naphthol on the resin side significantly reduces the thin film times when cured with different amine curing agents when compared to the thin film set times of neat resin cured with same curing agents.

The following examples describes the addition of 2-naphthol in flexible epoxy coatings.

### Example 10 (Formulated Curing Agent) (C1) (Compare Data with Just K-54)

A 3-neck ½L round bottom flask equipped with N₂ inlet, addition funnel and temperature probe was charged with 60 grams of Ancamide 910 polyamide curing agent, 10 grams of Ancamine 2716 modified polyamine curing agent, 15 grams of Ancamine 2914UF, 10 grams of Ancamine K54 and 5 grams of 2-naphthol and heated to 40 C and stirred until the 2-naphthol is completely dissolved in the curing agent blend.

### Example 11 (Formulated Curing Agent) (C2) (Compare with Only K-54)

A 3-neck ½L round bottom flask equipped with N₂ inlet, addition funnel and temperature probe was charged with 70 grams of Ancamide 910 polyamide curing agent, 10 grams of Ancamine 2716 modified polyamine curing agent, 10 grams of Ancamine K54 and 10 grams of 2-naphthol and heated to 40 C and stirred until the 2-naphthol is completely dissolved in the curing agent blend.

### Example 12 (Formulated Curing Agent) (C3)

A 3-neck ½L round bottom flask equipped with N₂ inlet, addition funnel and temperature probe was charged with 70 grams of Ancamide 910 polyamide curing agent, 10 grams of Priamine 1071 (Croda) curing agent, 10 grams of Ancamine 2914UF and 10 grams of 2-naphthol and heated to 40 C and stirred until the 2-naphthol is completely dissolved in the curing agent blend.

### Example 13 (Formulated Curing Agent) (C4)

A 3-neck ½L round bottom flask equipped with N₂ inlet, addition funnel and temperature probe was charged with 65 grams of Ancamide 910 polyamide curing agent, 20 grams of Ancamine 2914UF curing agent, 10 grams of Ancamine K54 and 5 grams of 2-naphthol and heated to 40 C and stirred until the 2-naphthol is completely dissolved in the curing agent blend.

### Example 14 (Formulated Curing Agent) (C5)

A 3-neck ½L round bottom flask equipped with N₂ inlet, addition funnel and temperature probe was charged with 60 grams of Ancamide 910 polyamide curing agent, 10 grams of curing agent composed of diethylenetriamine adducted with cresyl glycidyl ether (Epodil 742), 10 grams of Ancamine 2914UF and 20 grams of 2-naphthol and heated to 40 C and stirred until the 2-naphthol is completely dissolved in the curing agent blend.

### Example 15 (Formulated Curing Agent) (C6) (Compare with and without Naphthol)

A 3-neck ½L round bottom flask equipped with N₂ inlet, addition funnel and temperature probe was charged with 70 grams of Ancamide 910 polyamide curing agent, 10 grams of Tomamine PA-14, 10 grams of Ancamine K54 and 10 grams of 2-naphthol and heated to 40 C and stirred until the 2-naphthol is completely dissolved in the curing agent blend.

### Application and Performance Testing

Curing agent mixtures were prepared by mixing the components given in the above examples. with the epoxy component of standard bisphenol-A based epoxy resin of (Epon 828, DER 331 type), EEW 190 blended at 90:10 weight ratio with, unless specified otherwise. They were then mixed employing a stoichiometric level of 1:1 (amine: epoxy equivalents).

The following application testing was conducted on Formulated Curing agent examples C1-C5 and results are provided in Table 6.
1. Viscosity was measured using Brookfield Viscometer with Thermosel Accessory, Stand Alone (ASTM D-2196)
2. Thin film set times were measured at 6 mil thickness using ASTM D-5895.
3. Gel time was measured using ASTM D-2471
4. Shore D hardness was measured using ASTM C-2240
5. Die-C Tear strength was measured using ASTM 0-624
6. Trouser Tear strength was measured using ASTM D-1938
7. Adhesion to dry and damp concrete was measured using ASTM D-7234
   Damp concrete was prepared by submersing the concrete block in water half way for 24 hrs before application.
8. Tensile properties were measured using ASTM D-638.

TABLE 6

| Curing Agent | C1 | C2 | C3 |
|---|---|---|---|
| Curing agent Viscosity @ | 3200 | ND | ND |
| 25° C. | | | |
| Mix Viscosity @ 25° C. | 2683 | ND | ND |
| Gel Time (150 gram mix) | 26 | ND | ND |
| 25° C. | | | |
| TFST I/II/III @ 22° C. (min) | 110/195/255 | 110/235/295 | 150/230/330 |
| TFST I/II/III @5° C. | 180/315/405 | ND | ND |
| Shore D (24 h/48 h) | 75/75 | ND | ND |
| Die-C Tear Strength (PLI) | 699 | ND | ND |
| Trouser Tear Strength (PLI) | 199 | ND | ND |
| Adhesion to dry concrete | 715 | ND | ND |
| (psi) | | | |
| Adhesion to damp concrete | 642 | ND | ND |
| (psi) | | | |
| Tensile Strength (psi) | 3252 | ND | 1431 |
| Tensile Elongation (%) | 96 | ND | 60 |

TABLE 6

| Curing Agent | C4 | C5 | C6 |
|---|---|---|---|
| Curing agent | 4900 | ND | 2865 |
| Viscosity @ 25° C. | | | |
| (cP) | | | |
| Mix Viscosity @ | 3567 | ND | 2133 |
| 25° C. | | | |
| Gel Time (150 | 22 | ND | 30 |
| gram mix) 25° C. | | | |
| TFST I/II/III @ 22° C. | 120/165/230 | 135/195/280 | 145/250/310 |
| (min) | | | |
| TFST I/II/III @5° C. | 240/345/420 | ND | 360/510/705 |
| Shore D (24 h/48 h) | 75/75 | ND | 66/70 |
| Die-C Tear | 486 | ND | 298 |
| Strength (PLI) | | | |
| Trouser Tear | 347 | ND | 234 |
| Strength (PLI) | | | |
| Adhesion to dry | 1011 | ND | 987 (concrete |
| concrete (psi) | | | failure) |
| Adhesion to damp | ND | ND | 617 (concrete |
| concrete (psi) | | | failure) |
| Tensile Strength | ND | ND | 2105 |
| (psi) | | | |
| Tensile Elongation | ND | ND | 186 |
| (%) | | | |

### Application and Performance Testing

Curing agent mixtures were prepared by mixing the components given in the examples C6 were mixed with the epoxy component of standard bisphenol-F based epoxy resin of (DER 354 type resin), EEW 175 (R1), blended at 90:10 weight ratio with Epodil 748 (R₂), Ancarez 2364 (R₃) and a combination of Ancarez 2364 with Epodil 748 (R4), unless specified otherwise. They were then mixed employing a stoichiometric level specified (amine: epoxy equivalents).

The following application testing was conducted on Formulated Curing agent examples C₆ with Resin R₁-R₄ and results are provided in Table 7.
1. Viscosity was measured using Brookfield Viscometer with Thermosel Accessory, Stand Alone (ASTM D-2196)
2. Thin film set times were measured at 6 mil thickness using ASTM D-5895.
3. Gel time was measured using ASTM D-2471
4. Shore D hardness was measured using ASTM D-2240
5. Waterspot test—Internal test
6. Carbamation test—internal test
7. Pore resistance—Electrical Impedance Spectroscopic measurements on S412 Panels
8. Impact Test—on S412 Panels; Direct impact test ASTM-G14 and Reverse Impact test ASTM D2794
9. Abrasion resistance—ASTM D4060 CS 17 Wheels, 1 Kg
10. Tensile properties were measured using ASTM D-412C.
11. Dolly Pull off Test on ¼^{th} inch Hot roll Steel sand blasted substrate—ASTM D4541

TABLE 7
Application testing

| Performance details | | R1 | R2 | R3 | R4 |
|---|---|---|---|---|---|
| Stoichiometric Ratio | Resin:Curing | 1:1.45 | 1:1.4 | 1:1.3 | 1:1.25 |
| | Agent | | | | |
| Initial Mix Viscosity | in mPa · S | 2838 | 1619 | 3500 | 1931 |
| @ 25° C. | | | | | |
| Gel Time (for 150 g | in min. | 23.3 | 27.9 | 24.8 | 30.1 |
| Mass) @ 25° C. | | | | | |
| TFST (Stage 3) | in hours | 2.4/3.4/4.0 | 3.0/3.8/ | 2.6/3.6/ | 3.0/4.0/ |
| Phase I/II/III @ 25° C. | | | 4.8 | 4.0 | 4.6 |
| TFST (Stage 3) | in hours | 5.0/8.0/9.8 | 6.0/9.4/ | 5.6/9.0/ | 7.0/11.4/ |
| Phase I/II/III @ 5° C. | | | 11.0 | 10.6 | 12.4 |
| Water Spot | 1 day & 7 | 5.0/5.0 | 5.0/5.0 | 5.0/5.0 | 5.0/5.0 |
| RT Cure (1-5, 5 | days | | | | |
| Best) | | | | | |
| Carbamation Test | 1 day & 7 | 5.0/5.0 | 5.0/5.0 | 5.0/5.0 | 5.0/5.0 |
| RT Cure (1-5, 5 | days | | | | |
| Best) | | | | | |
| Shore D Hardeness | 1 day & 7 | 64/62 | 55/56 | 54/54 | 48/47 |
| | days | | | | |
| Pore Resistance, | After 24 h | 1.4 × 10⁵ | 3.7 × 10⁵ | 7.0 × 10⁵ | 1.1 × 10⁴ |
| EIS measurements | immersion in | | | | |
| | ohms | | | | |
| Direct Impact Test | in Cm · Kg | 186 | 186 | 186 | 186 |
| Reverse Impact test | in Cm · Kg | 186 | 186 | 186 | 186 |
| Abrasion test, CS17 | After 500 | 15.0/36.2 | 37.9/70 | 14.2/42.5 | 6.9/63.5 |
| wheels (1 kg) - Wt | and 1000 | | | | |
| loss in milligram | cycles | | | | |
| Tensile Strength | psi | 2520 | 1750 | 2080 | 1280 |
| Elongation | % | 158 | 170 | 183 | 173 |
| Young's Modulus | psi | 48000 | 14200 | 18900 | 6000 |
| Dolly Pull off | Adhesive to | 1550 | 1685 | 1548 | 2116 |
| | Dolly failure | | | | |
| | (YIZ) in psi | | | | |

1. A curing agent composition comprising (a) at least one naphthol or naphthol derivative represented by the structure (1) below: 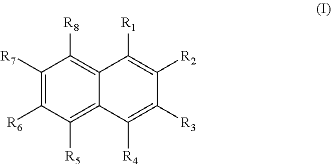
wherein R₁ and R₂ independently of each other=OH, H, C₁-C₁₀ alkyl, C₁-C₁₀ aryl, C₁-C₁₀ alkyl or aryl ether, NH₂, Cl, Br, I, NO₂, HSO₃, or X(CH₂)NHY with Y═C₁-C₁₀ alkyl, C₁-C₁₀ aryl, or a polyamine and X=Ph or C₁-C₄ alkyl; R₃-R₃═H, C₁-C₁₀ alkyl, C₁-C₁₀ aryl, C₁-C₁₀ alkyl or aryl ether, NH₂, Cl, Br, I, NO₂, or HSO₃; and wherein one of R₁ or R₂═OH; and
(b) at least one polyamine having three or more active amine hydrogens.

2. The composition of claim 1 wherein the at least one naphthol or naphthol derivative is represented by the structures (II)-(V) below: 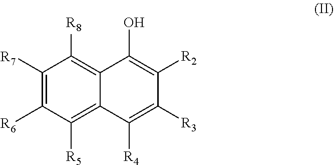
wherein R₂-R₃═H, C₁-C₁₀ alkyl, C₁-C₁₀ aryl, C₁-C₁₀ alkyl or aryl ether, NH₂, Cl, Br, I, NO₂, or HSO₃; 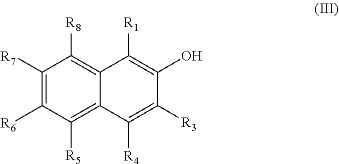
wherein R₁ and R₃—R₈═H, C₁-C₁₀ alkyl, C₁-C₁₀ aryl, C₁-C₁₀ alkyl or aryl ether, NH₂, Cl, Br, I, NO₂, or HSO₃; 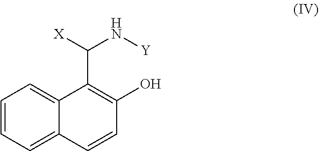
wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl; and 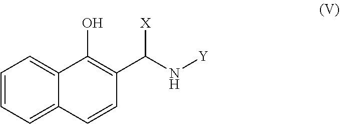
wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl.

3. The composition according to claim 2 wherein the at least one naphthol or naphthol derivative is selected from the group consisting of 4-methyl-1-naphthol, 2-methyl-1-naphthol,4-amino-3-methyl-1-naphthol, 4-methoxy-1-naphthol, 3-methoxy-2-naphthol, 5-methoxy-1-naphthol, 4-chloro-1-naphthol,1-chloro-2-naphthol, 1-bromo-2-naphthol, and 1-naphthol-4-sulfonic acid.

4. The composition according to claim 2 wherein the at least one naphthol derivative is obtained by a Mannich reaction wherein 1-naphthol or 2-naphthol is reacted with an aldehyde and an amine to form a Mannich base.

5. The composition according to claim 2 further comprising at least one compound selected from the group consisting of a boron trifluoride amine complex, 2,4,6-tri(dimethylaminomethyl)phenol, benzyldimethylamine, an imidazole, calcium nitrate, a carboxylic acid, salicylic acid, and sulfuric acid.

6. (canceled)

7. A method for producing a composition comprising the steps of (a) dissolving at least one naphthol or naphthol derivative in at least one polyamine to form a mixture; and (b) reacting the mixture with an epoxy resin component.

8. A method for producing a composition comprising the steps of (a) dissolving at least one naphthol or naphthol derivative in an epoxy resin component to form a mixture; and (b) reacting the mixture with at least one polyamine.

9. The method according to claim 7 wherein the at least one naphthol or naphthol derivative is represented by the structure (I) below: 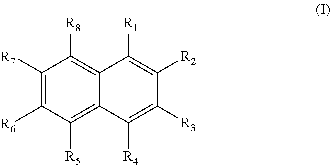
wherein R₁ and R₂ independently of each other=OH, H, C₁-C₁₀ alkyl, C₁-C₁₀ aryl, C₁-C₁₀ alkyl or aryl ether, NH₂, Cl, Br, I, NO₂, HSO₃, or X(CH₂)NHY with Y═C₁-C₁₀ alkyl, C₁-C₁₀ aryl, or a polyamine and X=Ph or C₁-C₄ alkyl; R₃-R₃═H, C₁-C₁₀ alkyl, C₁-C₁₀ aryl, C₁-C₁₀ alkyl or aryl ether, NH₂, Cl, Br, I, NO₂, or HSO₃; and wherein one of R₁ or R₂═OH.

10. The method of claim 9 wherein the at least one naphthol or naphthol derivative is represented by the structures (II)-(V) below: 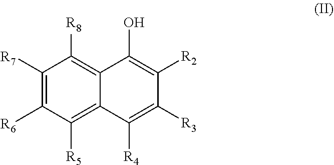
wherein R₂-R₃═H, C₁-C₁₀ alkyl, C₁-C₁₀ aryl, C₁-C₁₀ alkyl or aryl ether, NH₂, Cl, Br, I, NO₂, or HSO₃; 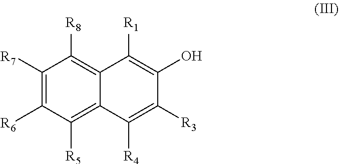
wherein R₁ and R₃—R₈═H, C₁-C₁₀ alkyl, C₁-C₁₀ aryl, C₁-C₁₀ alkyl or aryl ether, NH₂, Cl, Br, I, NO₂, or HSO₃; 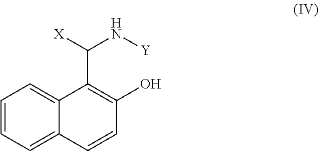
wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl; and 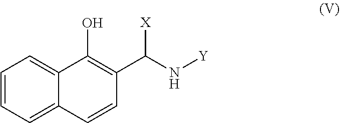
wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl.

11. The method according to claim 10 wherein the at least one naphthol or naphthol derivative is selected from the group consisting of 4-methyl-1-naphthol, 2-methyl-1-naphthol, 4-amino-3-methyl-1-naphthol, 4-methoxy-1-naphthol, 3-methoxy-2-naphthol, 5-methoxy-1-naphthol, 4-chloro-1-naphthol,1-chloro-2-naphthol, 1-bromo-2-naphthol, and 1-naphthol-4-sulfonic acid.

12. The method according to claim 10 wherein the at least one naphthol derivative is obtained by a Mannich reaction wherein 1-naphthol or 2-naphthol is reacted with an aldehyde and an amine to form a Mannich base.

13. A method of preparing hardened articles of manufacture comprising reacting the curing agent composition according to claim 2.

14. The method of claim 13, wherein the article is a coating, a construction product, a flooring product, or a composite product.

15. The method of claim 14, wherein the article is a coating.

16. The method of claim 15, wherein the coating is prepared at ambient temperature.

17. The method of claim 15, wherein the coating is prepared at a sub-ambient temperature as low as 0° C.

18. The method of claim 15 wherein the coating is a flexible epoxy coating.

19. A composition comprising the reaction product of:
(a) at least one naphthol or naphthol derivative represented by the structure (1) below: 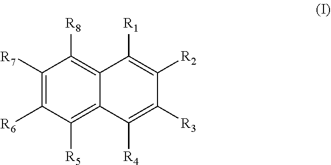
wherein R₁ and R₂ independently of each other=OH, H, C₁-C₁₀ alkyl, C₁-C₁₀ aryl, C₁-C₁₀ alkyl or aryl ether, NH₂, Cl, Br, I, NO₂, HSO₃, or X(CH₂)NHY with Y═C₁-C₁₀ alkyl, C₁-C₁₀ aryl, or a polyamine and X=Ph or C₁-C₄ alkyl; R₃-R₃═H, C₁-C₁₀ alkyl, C₁-C₁₀ aryl, C₁-C₁₀ alkyl or aryl ether, NH₂, Cl, Br, I, NO₂, or HSO₃; and wherein one of R₁ or R₂═OH;
(b) at least one polyamine having three or more active amine hydrogens; and
(c) an epoxy resin component comprising at least one multifunctional epoxy resin.

20. The composition of claim 19 wherein the at least one naphthol or naphthol derivative is represented by the structures (II)-(V) below: 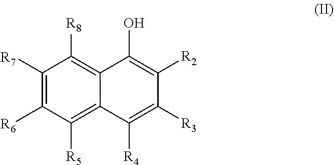
wherein R₂-R₃═H, C₁-C₁₀ alkyl, C₁-C₁₀ aryl, C₁-C₁₀ alkyl or aryl ether, NH₂, Cl, Br, I, NO₂, or HSO₃; 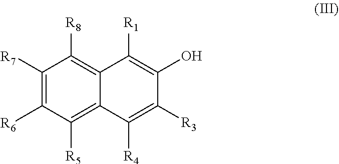
wherein R₁ and R₃—R₈═H, C₁-C₁₀ alkyl, C₁-C₁₀ aryl, C₁-C₁₀ alkyl or aryl ether, NH₂, Cl, Br, I, NO₂, or HSO₃; 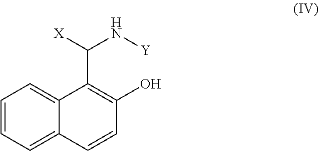
wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl; and 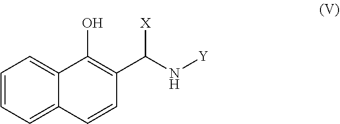
wherein Y═C₁-C₁₀ alkyl, C₁-C₁₀ aryl, or a polyamine selected from the group consisting of ethylenediamine, diethylenetriamine, triethylenetetramine, tetraethylenepentamine, N¹-(3-dimethylaminopropyl)propylenediamine, dimethylaminopropylamine, m-xylenediamine, and 4,4′-methylene-dicyclohexylamine, X=Ph or C₁-C₄ alkyl.

21. The method according to claim 8 wherein the at least one naphthol or naphthol derivative is represented by the structure (1) below: 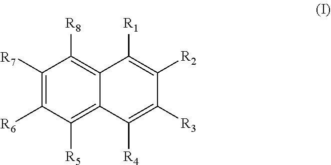
wherein R₁ and R₂ independently of each other=OH, H, C₁-C₁₀ alkyl, C₁-C₁₀ aryl, C₁-C₁₀ alkyl or aryl ether, NH₂, Cl, Br, I, NO₂, HSO₃, or X(CH₂)NHY with Y═C₁-C₁₀ alkyl, C₁-C₁₀ aryl, or a polyamine and X=Ph or C₁-C₄ alkyl; R₃-R₃═H, C₁-C₁₀ alkyl, C₁-C₁₀ aryl, C₁-C₁₀ alkyl or aryl ether, NH₂, Cl, Br, I, NO₂, or HSO₃; and wherein one of R₁ or R₂═OH.
